(19) United States
(12) Patent Application Publication
(10) Pub. No.: US 2022/0401407 A1
(43) Pub. Date: Dec. 22, 2022

(54) REGULATION OF THE SLEEP/WAKE CYCLE USING CANNABINOID COMPOUNDS

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/806,257

(22) Filed: Jun. 9, 2022

Related U.S. Application Data
(60) Provisional application No. 63/208,813, filed on Jun. 9, 2021.

(51) Int. Cl. A61K 31/352

(52) U.S. Cl. CPC A61K 31/352

(57) ABSTRACT

Methods of regulating or treating the sleep/wake cycle and related sleep disorders with cannabinoid compounds are described herein. The cannabinoid compounds can include one or more of cannabichromene (“CBC”), cannabicyclol (“CBL”), cannabinol (“CBN”), tetrahydrocannabivarin (“THCV”), cannabidol (“CBD”), (+)-cannabidiol (“(+)-CBD”), cannabigerol (“CBG”), and cannabigerol butyl (“CBG-C₄”) and can be antagonists to the receptors HCRTR1 and HCTR2. Compositions and articles including the cannabinoid compounds are further disclosed.

### CROSS-REFERENCE TO RELATED APPLICATIONS

The present application claims the priority benefit of U.S. provisional patent application Ser. No. 63/208,813, entitled REGULATION OF THE SLEEP/WAKE CYCLE USING CANNABINOID COMPOUNDS, filed Jun. 9, 2021, which is hereby incorporated herein by reference in its respective entirety

### TECHNICAL FIELD

The present disclosure generally relates to the use of cannabinoid compounds and related products to regulate the sleep/wake cycle of an individual. The cannabinoid compounds can act as agonists or antagonists to one or more human receptors that influence the sleep/wake cycle.

### BACKGROUND

The sleep/wake cycle refers to the cyclical nature of sleep and wakefulness necessary for daily functioning and refers to the pattern of time spent awake and asleep every 24 hours. In humans, the time is divided between approximately eight hours of sleep and 16 hours of wakefulness. Deviations from the sleep/wake cycle can cause a variety of sleep disorders ranging from minor impairment to severe conditions requiring medical treatment such as narcolepsy or insomnia. Regulation of the sleep/wake cycle is influenced by a variety of systems in the body including the locus coeruleus noradrenergic system. For example, activation of the orexin receptor system in the lateral hypothalamus can innervate the locus coeruleus and can increase wakefulness. Other receptors known to influence the sleep/wake cycle include dopamine receptors, such as DRD₅ receptors.

### SUMMARY

According to one embodiment, a composition or article includes one or more cannabinoid compounds, the composition or article regulating or treating a sleep disorder.

According to another embodiment, a method of regulating the sleep/wake cycle of an individual includes administering a therapeutically effective amount of one or more cannabinoid compounds.

### DETAILED DESCRIPTION

Methods and treatments of regulating the sleep/wake cycle are highly desirable to control sleep disorders and improve alertness, functioning, and general well-being. As can be appreciated, existing methods of regulating the sleep/wake cycle suffer from a variety of issues. For example, lifestyle and behavioral treatments are time-consuming and require considerable effort to enact meaningful change while known medical drugs suffer from issues such as addiction and side effects.

Compositions, articles, products, methods and treatments used to regulate the sleep/wake cycle using cannabinoid compounds are described herein. In certain embodiments, the cannabinoid compounds described herein can regulate (e.g., control) the sleep/wake cycle by acting as antagonists to two G-protein-coupled receptors (“GPCR's) in the orexin receptor system located in the lateral hypothalamus: hypocretin-orexin receptor 1 (“HCRTR1”) and hypocretin-orexin receptor 2 (“HCRTR2”) (also referred to as orexin receptor type 1 (“OX₁R”) and orexin receptor type 2 (“OX₂R”), respectively). Additionally, or alternatively, the cannabinoid compounds described herein can regulate the sleep/wake cycle by acting as an agonist to dopamine receptors, such as dopamine receptor D5 (DRD₅ receptors).

Prior experimental studies have indicated that antagonists to both HCRTR1 and HCRTR2 can increase the amount of Rapid Eye Movement (“REM”) sleep, induce sleep, and treat insomnia and narcolepsy. Attempts have been made to develop traditional small molecule chemistry drugs to act as antagonists for both HCRTR1 and HCRTR2 including almorexant, lemborexant, and nemorexant. The cannabinoid compounds described herein can achieve the same effect as these drugs with a significantly improved safety profile. As can be appreciated, by acting as antagonists to HCRTR1 and HCRTR2, the described cannabinoid compounds can mediate the orexin receptor system and decrease the levels of wakefulness and thereby promote sleep.

It has presently been discovered that only certain cannabinoid compounds exhibit an antagonistic effect on both of the orexin receptors HCRTR1 and HCRTR2. The cannabinoid compounds discovered to have an antagonist effect on both HCRTR1 and HCRTR2 are cannabichromene (“CBC”), cannabicyclol (“CBL”), cannabinol (“CBN”), tetrahydrocannabivarin (“THCV”), cannabidol (“CBD”), (+)-cannabidiol (“(+)-CBD”), cannabigerol (“CBG”), and cannabigerol butyl (“CBG-C₄”). Other cannabinoid compounds were discovered not to have an antagonist effect on HCRTR1 and HCRTR2 and would be expected to have no, or even possibly adverse effects, on regulation of the sleep/wake cycle.

For example, it has been discovered through a screening assay that cannabidiolic acid (“CBDA”), cannabidivarinic acid (“CBDVA”), cannabigerolic acid (“CBGA”), cannabidiorcol (“CBD-C1”), cannabidol-C₂ (“CBD-C2”), cannabigerolic acid butyl (“CBGA-C₄”), cannabigerovarinic acid (“CBGVA”), cannabichromenic acid (“CBCA”), and cannabicyclolic acid (“CBLA”) do not demonstrate meaningful activity against either HCRTR1 or HCRTR2 while cannabidivarin (“CBDV”) and cannabigerivarin (“CBGV”) demonstrate activity against only HCRTR1. Such cannabinoid compounds are not expected to be useful for the regulation of the sleep/wake cycle or treatment of any sleep disorders.

Prior to the present discovery, it was not appreciated that specific cannabinoid compounds could actively influence an individual's sleep/wake cycle. The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to regulate the sleep/wake cycle using only specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally-derived cannabinoid compounds offer benefits in regard to the sleep/wake cycle not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of regulating the sleep/wake cycle that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally extracted cannabinoid compounds are sufficiently pure and/or if any containments are sufficiently well understood.

In certain embodiments, selection of the cannabinoid compounds can be further influenced by additional receptor activity caused by the selected cannabinoid compounds. For example, the cannabinoid compounds described herein were further evaluated for DRD₅ receptor activity. As can be appreciated, activation of the DRD₅ receptor with an agonist has been linked to sleep inducement. Based on a GPCR assay, CBL and THCV have been identified as DRD₅ agonists.

In certain embodiments, the cannabinoid compounds used to treat sleep conditions by regulating the sleep/wake cycle can be CBL or THCV based on their dual antagonistic activity against HCRTR1 and HCRTR2 as well as their agonist activity against DRD₅. In certain embodiments, the compositions and methods described herein can include CBL or THCV, or both of CBL or THCV.

Generally, the sleep/wake cycle can be regulated by treatment with a therapeutically effective amount of one or more of CBC, CBL, CBN, THCV, CBD, (+)-CBD, CBG, and CBG-C4. In certain embodiments, each of CBC, CBL, CBN, THCV, CBD, (+)-CBD, CBG, and CBG-C4 can be included in a composition or article to regulate a sleep/wake cycle while in other embodiments, only a subset of CBC, CBL, CBN, THCV, CBD, (+)-CBD, CBG, and CBG-C4 can be included in such compositions and articles.

A therapeutic amount of the one or more cannabinoid compounds can vary depending on factors such as the desired effect of treatment, the duration of treatment, and the method of delivering the cannabinoid compounds to the subject. For example, to increase the duration of sleep may require a different amount, or dosage, of the cannabinoid compounds than the amount required to quickly induce sleep.

In certain embodiments, a therapeutic amount can be about 100 mg of the cannabinoid compounds or less; in certain embodiments, about 75 mg of the cannabinoid compounds or less; in certain embodiments, about 50 mg of the cannabinoid compounds or less; in certain embodiments, about 20 mg of the cannabinoid compounds or less; in certain embodiments, about 10 mg of the cannabinoid compounds or less; in certain embodiments, about 5 mg of the cannabinoid compounds or less; in certain embodiments, about 1 mg of the cannabinoid compounds or less; in certain embodiments, about 500 μg of the cannabinoid compounds or less; in certain embodiments, about 100 μg of the cannabinoid compounds or less; and in certain embodiments, about 500 pg of the cannabinoid compounds or less.

As can be appreciated, the relative concentration of the cannabinoid compounds can vary. For example, a beverage containing the cannabinoid compounds can have a smaller concentration of the cannabinoid compounds than a pill or capsule. In certain embodiments however, the total amount of the cannabinoid compounds can be the same between such two compositions and articles. In other embodiments, both the concentration and amount of cannabinoid compounds can vary between different compositions and articles.

In certain embodiments, the relative amounts of each of CBC, CBL, CBN, THCV, CBD, (+)-CBD, CBG, and CBG-C4 can vary in the compositions and articles described herein. For example, each individual cannabinoid compound (CBC, CBL, CBN, THCV, CBD, (+)-CBD, CBG, or CBG-C4) can vary from each other cannabinoid compound by about 1,000:1 to about 1:1,000. As can be appreciated, the amount and ratios of each of the cannabinoid compounds can be selected based on factors such as the method of delivery, the desired duration and type of regulation of the sleep/wake cycle, and individual factors such as the body weight of person consuming the cannabinoid compounds.

In certain embodiments, the compositions, articles, and methods described herein can be substantially or entirely free of cannabinoid compounds other than CBC, CBL, CBN, THCV, CBD, (+)-CBD, CBG, and CBG-C4 including, for example, substantially or entirely free of CBDA, CBDVA, CBGA, CBD-C1, CBD-C2, CBGA-C4, CBGVA, CBCA, CBLA and tetrahydrocannabinol (“THC”). In certain embodiments, the compositions and methods described herein can be substantially or entirely free of CBD, THC, CBDA, CBDVA, CBGA, CBD-C1, CBD-C2, CBGA-C4, CBGVA, CBCA, and CBLA. As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally-derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compound, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the cannabinoid compounds described herein can shift the time or duration of the sleep/wake cycle such as by inducing earlier sleep, on-demand sleep, longer sleep, or the type of sleep. For example, use of the cannabinoid compounds described herein can increase the duration of time spent in REM sleep or slow-wake sleep (also referred to as “deep sleep”). As can be appreciated, multiple benefits such as earlier induced sleep and deeper sleep states can also be achieved in certain embodiments.

In certain embodiments, the compositions, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds. As can be appreciated, it can be useful in certain embodiments, to release the cannabinoid compounds described herein using a delayed release mechanism, such as a delayed release pill, to regulate the bioavailable amounts of the cannabinoid compounds.

In certain embodiments, the cannabinoid compounds described herein can be useful to regulate not only the sleep/wake cycle but also treat various sleep disorders. For example, in certain embodiments, the cannabinoid compounds can be used to treat insomnia or narcolepsy.

In certain embodiments, the cannabinoid compounds described herein can regulate the sleep/wake cycle by inclusion in a composition or article. The composition or article can be consumed by, or be applied to, a person to regulate the sleep/wake cycle. Generally, the exact nature of the composition or article can vary widely.

For example, in certain embodiments, the cannabinoid compounds can be included in pills or capsules that can be taken quickly and efficiently on a regular or as needed basis (daily, with meals, etc.). As can be appreciated, pills and capsules can contain a number on inactive ingredients as known in the art such as dicalcium phosphate dehydrate, microcrystalline cellulose, stearic acid, silicon dioxide, croscarmellose sodium, magnesium stearate, and pharmaceutical glaze. Other known pills and capsules are also contemplated herein. As an additional example, a compressed chewable tablet can include a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), the cannabinoid compounds and, optionally, a lubricant (such as magnesium stearate, stearic acid, talc, and waxes), sweetening, coloring and flavoring agents, a surfactant, a preservative, and other ingredients. All of the ingredients, including the one or more cannabinoid compounds, are dry blended and compressed into a tablet. In certain embodiments, the tablet, pill, or capsule can be swallowed whole. In certain embodiments, the tablet, pill, or capsule can be a chewable capsule.

In certain embodiments, the cannabinoid compounds can alternatively be administered to individuals via food products and other comestibles. By way of illustration and not as a limitation, the selected cannabinoid compounds can be incorporated into a beverage, a “smoothie” (fruit, vegetable, nut oil, or yogurt based), a frozen desert (e.g., ice cream or sorbet), a food bar, a nutrition bar, a dressing, a snack, into a flour- or flour-alternative-based product, a rice-based product, pastes, gels, powders, gums, etc. In certain embodiments, the food product can be a gummy. Incorporation into food products can facilitate consumption of the cannabinoid compounds and increase palatability.

As can be appreciated, the exact nature of the food article can influence the bioavailability of the cannabinoid compounds. For example, a cannabinoid included in a large food article may take more time to become bioavailable than the same amount of cannabinoid compounds in a single pill or capsule. Generally, the remainder of the composition or article can constitute any suitable non-bioactive component such as filler, food, or water.

In certain embodiments, the compositions or articles including the cannabinoid compounds described herein can include indicia and/or packaging to convey to end users the amount of the cannabinoid compounds contained therein. For example, a small nutrient bar may be individually labeled and packaged to express to the end user that only a single bar should be consumed.

As will be appreciated, a wide variety of different compositions and articles can be prepared which include the one or more cannabinoid compounds of the present disclosure including compositions and articles not listed here. All such compositions and articles are contemplated herein as they are within the ordinary skill of artisans based on the guidance provided in the present disclosure.

In certain embodiments, the cannabinoid compounds described herein can alternatively be externally delivered to the body through use of product formed from a personal care composition. Examples of suitable personal care compositions include emulsions, suspensions, liquids, pastes, gels, ointments, creams, sprays, powders, films, and patches. For example, in certain embodiments, the cannabinoid compounds can be applied to a person through a patch applied to the skin containing the cannabinoid compounds dissolved in a suitable solvent such as an alcohol.

Generally, all of the compositions and articles described herein can be manufactured and produced as known in the art. For example, in certain embodiments, the cannabinoid compounds can be dissolved in a suitable solvent such as an alcohol or oil and then added to the composition or article.

### Examples

To evaluate the role specific cannabinoid compounds may play on the sleep/wake cycle, a GPCR reactivity assay was performed to determine the reactivity of 19 cannabinoid compounds to the orexin receivers HCRTR1 and HCRTR2. The evaluated cannabinoid compounds were: CBN, THCV, CBDVA, CBG, CBL, CBC, CBDV, CBDA, CBD, CBGA, (+)-CBD, CBG-C4, CBD-C1, CBD-C2, CBGV, CBGA-C4, CBGVA, CBCA, and CBLA. Orexin A was used as the control for HCRTR1 and HCRTR2.

To perform the GPCR reactivity assay, a commercial GPCR assay, PathHunter® β-Arrestin from Eurofins DiscoverX Products (Fremont, Calif.), was used. In the PathHunter® β-Arrestin GPCR assay, an inactive peptide fragment is fused to the targeted GPCR receptor and a complementary peptide fragment is fused to β-arrestin. At activation of the GPCR receptor and recruitment of β-arrestin, complementation of the peptide fragments occurs and restores β-galactosidase activity. The amount of β-galactosidase activity is then measurable using chemiluminescent reagents.

A total of four assays were run. Agonist assays were run for each of HCRTR1 and HCRTR2 and antagonist assays were run for HCRTR1 and HCRTR2. In each of the two agonist assays, PathHunter® cell lines were removed from a freezer stock and seeded at a volume of 20 μL into white walled, 384-well microplates and incubated at 37° C. Each cell was incubated with a sample to induce a response and then diluted to generate a 5× sample in assay buffer. 5 μL of the 5× sample was then added to cells and incubated at 37° C. for 90 or 180 minutes. The final assay concentration was 1%. The assay signal was generated through addition of 12.5 μL or 15 μL (50% V/V) of a detection reagent cocktail followed by a one hour incubation time at room temperature. Microplates were read following signal generation with a PerkinElmer Envision™ (PerkinElmer Inc., Waltham, Mass.) for chemiluminescent signal detection. Reactivity was analyzed using the CBIS data analysis suite (ChemInnovation, CA) where RLU refers to the raw measured values. The percent activity was then calculated using the formula: % Activity=100%*(mean RLU of test sample−mean RLU of control)/(mean max control ligand−mean RLU of vehicle control).

In each of the two antagonist assays, PathHunter® cell lines were removed from a freezer stock and seeded at a volume of 20 μL into white walled, 384-well microplates and incubated at 37° C. Each cell was pre-incubated with an antagonist followed by an agonist challenge at the EC80 concentration. Cells were then diluted to generate a 5× sample in assay buffer. 5 μL of the 5× sample was then added to cells and incubated at 37° C. for 30 minutes. Finally, 5 μL of 6×EC80 agonist in assay buffer were added to the cells and incubated at 37° C. for 90 minutes or 180 minutes. The assay signal was generated through addition of 12.5 μL or 15 μL (50% V/V) of a detection reagent cocktail followed by a one hour incubation time at room temperature. Microplates were read following signal generation with a PerkinElmer Envision™ (PerkinElmer Inc., Waltham, Mass.) for chemiluminescent signal detection. Reactivity was analyzed using the CBIS data analysis suite (ChemInnovation, CA) where RLU refers to the raw measured values. The percent inhibition was calculated using the formula: % Inhibition=100%*(1−mean RLU of test sample−mean RLU of control)/(mean RLU of EC80 control−mean RLU of vehicle control).

The results of the GCPR reactivity screen are depicted in Table 1. For agonist activity, the percent activity is depicted while for antagonist activity, the percent inhibition is depicted.

TABLE 1

| | (+)-CBD | CBD | CBDV | CBD-C2 | CBD-C1 | CBG | CBG-C4 | CBGV | CBC | CBL |
|---|---|---|---|---|---|---|---|---|---|---|
| HCRTR1 | 3% | 1% | 0% | 2% | 3% | 3% | 4% | 4% | 0% | 2% |
| (Agonist) | | | | | | | | | | |
| HCRTR2 | 8% | 5% | 0% | 1% | 1% | 4% | 6% | 2% | 3% | 5% |
| (Agonist) | | | | | | | | | | |
| HCRTR1 | 97% | 96% | 61% | 20% | 12% | 98% | 84% | 44% | 98% | 96% |
| (Antagonist) | | | | | | | | | | |
| HCRTR2 | 80% | 85% | 18% | 13% | 9% | 68% | 45% | 12% | 96% | 93% |
| (Antagonist) | | | | | | | | | | |

| | CBN | THCV | CBDA | CBDVA | CBGA | CBGA-C4 | CBGVA | CBCA | CBLA |
|---|---|---|---|---|---|---|---|---|---|
| HCRTR1 | 1% | 2% | 1% | 0% | 1% | 3% | 0% | 3% | 2% |
| (Agonist) | | | | | | | | | |
| HCRTR2 | 7% | 6% | 0% | 0% | 1% | 1% | 1% | 1% | 0% |
| (Agonist) | | | | | | | | | |
| HCRTR1 | 95% | 89% | 2% | 7% | 8% | 2% | 3% | 12% | 7% |
| (Antagonist) | | | | | | | | | |
| HCRTR2 | 94% | 89 | 10% | 10% | 14% | 10% | 12% | 17% | 15% |
| (Antagonist) | | | | | | | | | |

As depicted in Table 1, none of the 19 evaluated cannabinoid compounds acted as an agonist for either HCRTR1 or HCRTR2. CBDV and CBGV acted as antagonists for HCRTR1 but did not act as antagonists for HCRTR2. CBC, CBL, CBN, THCV, CBD, (+)-CBD, CBG, and CBG-C4 acted as an antagonist for both HCRTR1 and HCRTR2.

Table 2 depicts the results of evaluating the cannabinoid compounds to act as an agonist for the dopamine receptor DRD5. The same experimental procedure as used for HCRTR1 and HCRTR2 agonist testing (Table 1) was followed with the substitution of dopamine as the control for the DRD5 receptor.

TABLE 2

| | (+)-CBD | CBD | CBDV | CBD-C2 | CBD-C1 | CBG | CBG-C4 | CBGV | CBC | CBL |
|---|---|---|---|---|---|---|---|---|---|---|
| DRD₅ (Agonist) | 9% | 27% | 7% | 1% | 0% | 9% | 6% | 6% | 22% | 37% |

| | CBN | THCV | CBDA | CBDVA | CBGA | CBGA-C4 | CBGVA | CBCA | CBLA |
|---|---|---|---|---|---|---|---|---|---|
| DRD₅ (Agonist) | 3% | 52% | 5% | 4% | 0% | 0% | 0% | 1% | 0% |

As depicted by Table 2, cannabinoid compounds had a diverse set of responses to the DRD₅ GPCR receptor with THCV and CBL exhibiting stronger agonist effects on DRD₅.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. A composition or article comprising one or more cannabinoid compounds, the composition or article regulating or treating a sleep disorder.

2. The composition or article of claim 1, wherein the one or more cannabinoid compounds comprise cannabichromene (“CBC”), cannabicyclol (“CBL”), cannabinol (“CBN”), tetrahydrocannabivarin (“THCV”), cannabidol (“CBD”), (+)-cannabidiol (“(+)-CBD”), cannabigerol (“CBG”), and cannabigerol butyl (“CBG-C₄”).

3. The composition or article of claim 1, wherein the one or more cannabinoid compounds are each hypocretin-orexin receptor 1 (HCRTR1) and hypocretin-orexin receptor 2 (HCRTR2) antagonists.

4. The composition or article of claim 1, wherein the one or more cannabinoid compounds comprise cannabicyclol (“CBL”) and tetrahydrocannabivarin (“THCV”).

5. The composition or article of claim 1, wherein the one or more cannabinoid compounds are dopamine receptor D5 (DRD₅) agonists.

6. The composition or article of claim 1 comprises about 10 grams or less of the one or more cannabinoid compounds.

7. The composition or article of claim 1 is a food or beverage.

8. The composition or article of claim 1 is a pill or capsule.

9. The composition or article of claim 8 is a delayed-release pill or capsule.

10. The composition or article of claim 1 is a powder or liquid.

11. The composition or article of claim 1 is a personal care product.

12. The composition or article of claim 1, wherein the one or more cannabinoid compounds are non-horticulturally derived cannabinoid compounds.

13. A method of regulating the sleep/wake cycle of an individual, the method comprising administering a therapeutically effective amount of one or more cannabinoid compounds.

14. The method of claim 13, wherein the one or more cannabinoid compounds comprise cannabichromene (“CBC”), cannabicyclol (“CBL”), cannabinol (“CBN”), tetrahydrocannabivarin (“THCV”), cannabidol (“CBD”), (+)-cannabidiol (“(+)-CBD”), cannabigerol (“CBG”), and cannabigerol butyl (“CBG-C₄”).

15. The method of claim 13 treats narcolepsy or insomnia.

16. The method of claim 13 normalizes the sleep/wake cycle.

17. The method of claim 13 is a treatment to issues caused by a non-normal sleep/wake cycle.

18. The method of claim 13, wherein the one or more cannabinoid compounds are non-horticulturally derived cannabinoid compounds.
